(19) United States
(12) Patent Application Publication
(10) Pub. No.: US 2026/0301888 A1
(43) Pub. Date: Oct. 1, 2026

(54) DATA ANALYSIS TECHNIQUES AND APPLICATIONS

(71) Applicant: PRESENTIENT TECHNOLOGIES LIMITED, Notting Hill Library, London (GB)

(72) Inventors: Tom COATES, London (GB); Dan NICOLAU, Notthingham Nottinghamshire (GB)

(21) Appl. No.: 19/480,665

(22) PCT Filed: May 10, 2024
(86) PCT No.: PCT/GB2024/051219

§ 371 Date: Oct. 31, 2025

(30) Foreign Application Priority Data
May 10, 2023 (GB) 2306913.1

(51) Int. Cl. G16H 10/20

(52) U.S. Cl. CPC G16H 10/20

(57) ABSTRACT

Disclosed is a method for generating a medical trial conclusion with respect to at least some participants in a medical trial, where medical trial data of the trial participants has been collected and the medical trial data collected comprises a data set pertaining to a first type of participant medical impact. The method comprises computer implemented analysis of the collected medical trial data of the medical trial participants. The analysis is to identify a first participant medical impact group by performing statistical clustering analysis for the medical trial participants. The statistical clustering analysis is in terms of participant medical impact as determined in accordance with the data set pertaining to the first type of participant medical impact. The first participant medical impact group corresponds to a statistical cluster so determined. The method further comprises generating a medical trial conclusion comprising an indication of the first participant medical impact group.

### TECHNICAL FIELD

The present disclosure relates to data analysis techniques and conclusions that may be drawn from them. The disclosure may have particular application to analysing medical trial data, but this is not intended to be limiting since the disclosure may have application to data analysis in other fields (e.g. performance and/or fault/failure analysis of a mechanical system or human behavioural analysis).

### BACKGROUND

Many existing data analysis approaches and techniques are relatively unrefined, which may lead to delayed sub-optimal, inaccurate or incomplete conclusions being drawn. Data may, for example, be underutilised and/or misinterpreted as a consequence of a data analysis approach which:
is non-adaptive;
makes assumptions which are inaccurate or represent over simplifications;
are predicated on conditions which limit the conclusions that can be drawn;
are too general/insufficiently detailed to provide more/all potentially useful information that might be generated from the data; and/or
are limited by factors such as imagination and/or pre-conceived concepts and/or time and/or timing.

Taking by way of example approaches to data analysis in medical trials, the medical trials themselves often involve groups of participants treated in different ways (e.g. the administration of different drugs and/or different dosages etc). Data is collected on these participants with a view to monitoring efficacy and side effects, often at least in part by comparing the effect on groups which are treated in different ways.

Such trials will usually have predetermined statistical conditions for success and failure of the whole trial (e.g. efficacy or not of the treatment). For instance, the data could be required to show for the participants on average that the treatment has a statistically significant benefit (relative to, say, placebo) and that the probability of this apparent benefit being the result of chance rather than the treatment is below a threshold percentage. In effect, the mean and standard deviation of the participant data is considered, which is a coarse approach liable to miss important nuances. Further, the trial must be completed for the results to be considered valid.

This rigidity and coarseness of this approach is designed at least in part to compensate for relatively superficial analysis of the data gathered. Known limitations in the data analysis techniques mean that it may be preferable to lay out pre-determined, rigid, coarse and/or conservative success and failure criteria. In this way, the potential for erroneous conclusions resulting from incomplete and/or relatively unsophisticated data analysis may be better avoided. Nonetheless, taking this approach, rather than improving the data analysis techniques, may lead to useful information (e.g. local successes embedded within an overall failure) not being recognised or even sought, wasting of resources and/or sub-optimal outcomes for medical trial participants and/or patients more generally. For instance, success for some trial participants and the reasons for this may not be recognised and/or may be ignored where overarching failure criteria for the trial have been fulfilled and/or insufficiently sophisticated analysis of the data is performed. Further, it may be that a trial is completed, despite its outcome being obvious or at least highly predictable well ahead of its completion, to the potential detriment of participant and patient outcomes and/or efficiency.

Although some attempts are currently made to analyse medical trial data in greater depth, these are limited in terms of their techniques, frequency and scope. For instance, periodic reviews are conducted throughout a trial e.g. at four month intervals. These reviews generally focus on identifying relatively obvious macro-trends which may need to be addressed, e.g. those related to safety, including side effects and instances of unexpected patient death or other adverse outcomes. At the end of a trial, some attempts may be made to segment participants into meaningful groups in order to better understand the reason for the observed medical impacts, but these approaches rely on the experienced intuition of a human to propose a manner in which the participants may be segmented into two groups. Using this as a basis, a check is performed to establish whether the proposed segments are meaningful. This is an inherently limited process in terms of its scope, depth and reliability, being very much dependent on the quality and sophistication of the proposal as well as being time consuming and error-prone.

It is an object of embodiments of the invention to at least mitigate one or more of the problems present in the prior art.

### SUMMARY OF THE INVENTION

According to an aspect of the invention, there is provided a method optionally for generating a medical trial conclusion optionally with respect to at least some participants in a medical trial, where medical trial data of the trial participants has optionally been collected and the medical trial data collected optionally comprises a data set optionally pertaining to a first type of participant medical impact, the method comprising:
optional computer implemented analysis of the medical trial data collected for the medical trial participants optionally to identify a first participant medical impact group optionally by performing statistical clustering analysis for the medical trial participants optionally in terms of participant medical impact optionally as determined in accordance with the data set optionally pertaining to the first type of participant medical impact, the first participant medical impact group optionally corresponding to a statistical cluster so determined; and
optionally generating a medical trial conclusion optionally comprising an indication of the first participant medical impact group.

By using statistical clustering, medical trial participants may be more accurately and usefully grouped, potentially thereby giving a more accurate model of reality on which to base further actions.

The indication of the first participant medical impact group may indicate the medical trial participants found to be in the first participant medical impact group, the size of the group (e.g. number of participants) and/or character of the group in terms of the nature of the medical impact that characterises it. Thus for instance, in the case that the first type of participant medical impact is tumour size reduction, the indication of the first participant medical impact group might comprise a corresponding list of medical trial participants, an indication that the first participant medical impact group represents 30% of the medical trial participants and is characterised by a relatively large tumour size reduction.

Where the statistical clustering analysis reveals one or more statistical clusters in addition to that of the first participant medical impact group, the medical trial conclusion may comprise indication of additional participant medical impact groups corresponding to one, some or all of the additional statistical clusters. Thus for instance, the medical trial conclusion may comprise an indication of a group for which there has been a small or negligible tumour size reduction and a group for which there has been negative tumour size reduction (i.e. the tumours have actually grown, on average). Further discussion below is provided, for simplicity, in the context of the first participant medical impact group, but it is envisaged that all features using or applied to the first participant medical impact group might alternatively or additionally be applied to one or more of the additional participant medical impact groups e.g. a second participant medical impact group.

Statistical clusters may only be identified as a group (e.g. the first participant medical impact group), if the cluster is determined to be statistically significant. Statistical significance may be tested using conventional techniques. Even where a cluster has few or even only one participant, it is possible to conduct analysis for statistical significance. In some cases the paucity of data may mean that there is an outcome of “not significant”, but in other cases, where significance is present, significance determination techniques may identify this even given the paucity of data.

Each type of participant medical impact is a different discernible (e.g. measurable) consequence for participants potentially/apparently resulting from or contributed to by application of an intervention of the medical trial (for instance treatment using a drug). Non-limiting examples of types of participant medical impacts include an indicator of efficacy of a treatment, (e.g. tumour size in the case of a cancer drug, microbial count or infection status in the case of an antibiotic, or erythrocyte sedimentation rate in the case of an anti-inflammatory agent) indicators of side effects (e.g. headache, vomiting, rash, fatigue or neurological disturbance etc) and death or other adverse event. At least some types of participant medical impact may be quantified (e.g. in terms of value/category/duration/severity/instances/degree etc). Such quantification could be through a single data point or value, or a data point or value series collected over time. Further, a data point or value recording and/or quantifying a type of participant medical impact could be discrete (i.e. having a binary value) or continuous (having a value selected from a continuum of possibilities) in nature. As will be appreciated, the absence of a medical impact may be treated as a valid quantification/record for a type of medical impact.

It is likely that the participants of a medical impact group discovered (for instance the first participant medical impact group) will have substantial commonality of participant medical impact. Any participant medical impact groups identified will correspond to a respective statistical cluster. It should be noted that the clustering process as applied to participant medical impact may give rise to at least some clusters that represent only a single participant (i.e. that a participant is their own cluster and from a grouping point of view is an outlier).

In some embodiments, the medical trial data collected comprises at least one data set each pertaining to a respective type of participant trait, and the method comprises:
analysing the collected medical trial data of the medical trial participants to identify one or more first participant traits represented within the first participant medical impact group, as determined in accordance with the at least one data set each pertaining to a respective type of participant trait; and
where one or more first participant traits are identified, generating the medical trial conclusion to comprise an indication of at least one of the first participant traits.

It may be, for instance, that first participant traits determined to be statistically significant or representing over a threshold percentage of the medical trial participants in the first participant medical impact group, or representing over a threshold percentage of all medical trial participants having the relevant participant trait are indicated in the medical trial conclusion.

The indication of each first participant trait may indicate the medical trial participants in the first participant medical impact group having the trait, the number of medical trial participants in the first participant medical impact group having the trait and/or the trait itself. Thus for instance, in the case that a type of participant trait is a BRAF gene mutation or otherwise, the indication of the first participant trait might comprise an indication that 90% of the first participant medical impact group comprises medical trial participants having a BRAF gene mutation.

In some embodiments, the analysing of the collected medical trial data of the medical trial participants to identify one or more first participant traits represented within the first participant medical impact group is computer implemented.

In some embodiments, the medical trial conclusion is generated to comprise an indication that individuals having a trait or traits consistent with one or more of the first participant traits are more likely to experience medical impact associated with the first participant medical impact group.

Types of participant trait are types of discernible (e.g. measurable) characteristic or circumstance of participants which are at least potentially relevant to the medical trial (e.g. to participant medical impact. Non-limiting examples of types of participant trait include an indicator of health status or information, (e.g. blood pressure, blood oxygenation, body mass index, disease stage or past medical event etc) personal characteristic or characteristics, (e.g. age, gender, ethnicity, height or genetic characteristic etc) personal contextual information, (e.g. location of residence, occupation, whether or not he/she has children, degree of exposure to a toxin etc) whether the participant is in a treatment or placebo arm of the medical trial and/or which treatment arm of the medical trial the participant is in. At least some types of participant trait may be quantified (e.g. in terms of value/category/duration/severity/instances/degree etc). Such quantification could be through a single data point or value, or a data point or value series collected over time. Further, a data point or value recording and/or quantifying a type of participant trait could be discrete (e.g. having a binary or categorical value) or continuous (having a value selected from a continuum of possibilities) in nature. As will be appreciated, non-expression/absence/applicability of a participant trait may be treated as a valid quantification/record of a type of participant trait. Further, such data points or values may apply to a pre-medical trial state (e.g. measured/collected prior to commencement of the medical trial), may apply to an in trial or post trial state or may span multiple such states (e.g. pre- and in-trial states with data points for the type of participant trait being collection on multiple occasions prior to and during the medical trial).

In accordance with the approach, a better and/or more complete understanding of impacts such as effectiveness may be achieved than would otherwise be possible. In particular, participants may be grouped by both impact and trait such that traits which may play a role in producing an impact may be identified. This may lead to different assessment and outcomes for the participants in the medical trial and/or for others who may be categorised in terms of their traits. Further, the method may indicate the likely impact and/or result of the medical trial and may lead to adjustments to the trial and/or may influence the design of other medical trials (e.g. future medical trials).

Where no first participant medical impact group is identified, the generated medical trial conclusion may be to this effect. Where no first participant trait is identified, the generated medical trial conclusion may be to this effect and/or may comprise another conclusion as discussed further below. A similar approach may be taken in respect of all such negative findings as will be apparent in the context of additional/alternative analysis possibilities as discussed further below, mutatis mutandis.

In some embodiments, the medical trial conclusion comprises an indication that individuals having a trait or traits in common with medical trial participants in the identified first participant medical impact group are more likely to experience medical impact associated with the first participant medical impact group in proportion to the prevalence of the relevant trait or traits as represented in the identified first participant medical impact group.

Generating such a conclusion may be particularly useful where despite one or more first participant traits being identified, none is found to be statistically significant and/or a significant determinant/indicator of medical impact. In this case, the first participant medical impact group may be considered a functional group, in that the participants in the functional group have similarity in terms of the first participant medical impact but do not have a significant link in terms of the specific data set or sets used each pertaining to respective types of participant traits. In such cases, it may nonetheless be informative to understand traits and their proportions present in the functional group. For instance, such information might be indicative of traits corresponding to risk markers and/or might indicate those with greater potential to be responders in terms of efficacy in the medical trial and/or future medical trials. Thus, such a medical trial conclusion may be particularly relevant where no statistically significant first participant trait is identified. Nonetheless, it may still form part of the medical trial conclusion in cases where one or more first participant traits which are statistically significant are identified, in which case it might complement the indication that individuals having traits consistent with an identified first participant trait are more likely to experience medical impact associated with the first participant medical impact group.

In some embodiments the medical trial data collected comprises a data set pertaining to a second type of participant medical impact.

In some embodiments, the method comprises:
computer implemented analysis of the collected medical trial data of the medical trial participants in the identified first participant medical impact group to identify a first participant medical impact sub-group within that first participant medical impact group, by performing statistical clustering analysis for the participants in the identified first participant medical impact group in terms of participant medical impact as determined in accordance with the data set pertaining to a second type of participant medical impact, the first participant medical impact sub-group corresponding to a statistical cluster so determined;
and where the first participant medical impact sub-group has been identified, the medical trial conclusion comprises an indication of the first participant medical impact sub-group.

In some embodiments, the medical trial conclusion comprises an indication that individuals having a trait or traits in common with participants in the identified first participant medical impact sub-group are more likely to experience medical impact associated with the identified first participant medical impact sub-group in proportion to the prevalence of the relevant trait or traits as represented in the identified first participant medical impact sub-group.

Thus, the method is to some degree repeated but for another type of participant medical impact. Consequently there may be a hierarchy of groups and sub-groups which may be identified. Accordingly, a better and/or more complete understanding of impacts may be achieved than would otherwise be possible. In particular, a conclusion may be generated such that an identified participant medical impact group is further sub-divided in accordance with another type of medical impact. Thus for instance, a group consisting of medical trial participants for whom a treatment (such as a drug) has similar effectiveness may be identified by the method and may be further analysed for differences in side effects.

In some embodiments, the method comprises:
analysing of the collected medical trial data of the medical trial participants to identify one or more second participant traits represented within the identified first participant medical impact sub-group, as determined in accordance with the at least one data set each pertaining to a respective type of participant trait; and
where one or more second participant traits are identified, generating the medical trial conclusion to comprise an indication of at least one of the second participant traits.

Thus, the first participant medical impact sub-group may itself be analysed in terms of participant traits.

In some embodiments, the analysing of the collected medical trial data of the medical trial participants to identify one or more second participant traits represented within the first participant medical impact group is computer implemented.

In some embodiments, the medical trial conclusion is generated to comprise an indication that individuals having a trait or traits consistent with one or more of the second participant traits are more likely to experience medical impact associated with the first participant medical impact sub-group.

As will be appreciated, further iterations are possible to extend the hierarchy and thereby give further output analysis detail.

In some embodiments, performing the statistical clustering analysis comprises calculating a distance metric between each of the participants in the medical trial and each of the other participants in the medical trial, each distance metric indicating a relative degree of similarity of the respective pair of medical trial participants in respect of the data set pertaining to the first type of participant medical impact. The distance metrics may be considered to form a network indicating the relationships between each of the participants in the medical trial in terms of similarity and differences as indicated by the data set pertaining to the first type of participant medical impact. This may form the basis of determining any clusters in to which the participants in the medical trial may be placed.

Where each medical trial participant has a single data point or value to represent its data in the data set pertaining to the first type of participant medical impact, the distance metric between each pair of participants may be the difference in their respective data points or values. Where however each medical trial participant has a time series of data points or values to represent its data in the data set pertaining to the first type of participant medical impact, an alternative approach may be taken to determining the relevant distance metric.

In some embodiments, calculating each of the distance metrics comprises calculating a correlation coefficient between the respective pair of participants in the medical trial. This approach may be appropriate where each medical trial participant has a time series of data points or values to represent its data in the data set pertaining to the first type of participant medical impact. The correlation coefficient may be calculated in accordance with the equation:

$${r}_{xy} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} \qquad Equation 1$$

Where r_{xy} is the correlation coefficient, xᵢ and yᵢ are instances of the time series of data points or values for participant x and participant y, respectively) and the barred x and y are mean values for the time series of data points or values for participant x and participant y respectively.

In some embodiments, calculating the distance metrics comprises:
generating a first eigenspectrum of a correlation matrix comprising the correlation coefficients;
generating a second eigenspectrum of a random matrix having the same dimensions as the correlation matrix;
removing eigenvalues of the second eigenspectrum from the first eigenspectrum;
generating a final matrix using the remaining eigenvalues and using these as the distance metrics.

This approach may be a significant improvement over the prior art because, in general, the majority (e.g. 90% or more) of the eigenvalues and associated eigenvectors of a raw correlation matrix may be associated with noise, and therefore the bulk of the raw correlation matrix entries may be indistinguishable from pure noise. Consequently, mitigating or reducing the effect of these eigenvalues may be significant in terms of clustering accuracy.

The random matrix may be selected to also have the same general mathematical properties (e.g. of the same size, symmetric and/or being positive semi-definite) as the correlation matrix.

In some embodiments, determination of the statistical clusters comprises using an algorithm that, given the distance metrics, seeks to maximise a function which indicates the degree of assortative grouping, modelling a baseline clustering configuration as a starting point and making random sequential adjustments to the configuration such that in each case the clusters are redefined so that one participant at a time is no longer in one cluster and is instead in another, analysing the effect of each adjustment on the function and where the function is larger, retaining the relevant adjustment and where the function is smaller, reversing the relevant adjustment. This process (i.e. random sequential adjustments which are retained or reversed as appropriate) may be continued until substantially no further increases in the function are achieved. Thus, where an adjustment causes distant data points to be defined as being in the same cluster, the function would tend to be smaller and so the adjustment would tend to be reversed and vice versa. The method may therefore divide participants into groups so as to increase internal group connectedness relative to connectedness between groups. This approach may be well suited to large data sets and/or diverse data set types (as may occur in the case of a medical trial). Specifically, the methodology allows for efficient clustering analysis despite it being difficult/inappropriate to make starting assumptions concerning the clustering of the data given its potential diversity of types. It does this by allowing for initial randomness in the baseline whilst ensuring a relatively rapid convergence on a solution which will approximate an optimal solution. The approach therefore gives advantages in terms of reducing processing and time required. Additionally, the approach may offer improved results by comparison with simply performing correlation analysis on the distance metrics calculated, which would introduce global noise in the process, due to the mathematical operations inherent in the computation of correlation values rather than structure/signals in the data itself.

In some embodiments, the function is of the form:

$$Q ( \vec{σ} ) = \frac{1}{{A}_{Tot}} {\sum }_{i , j} [ {A}_{i , j} - 〈 {A}_{i , j} 〉 ] δ ( {σ}_{i} , {σ}_{j} ) \qquad Equation 2$$

Where Q is the function to be maximised indicating degree of assortative grouping, A_{Tot} is the sum of the eigenvalues of the final matrix, A_{i,j} are the eigenvalues of the final matrix, <A_{i,j}> is the expected eigenvalue in the final matrix given an assumption of the matrix representing a random graph and a indicates membership of a given cluster where 1 indicates membership and 0 non-membership.

Such a function may assist in more accurately identifying clusters in a heterogenous and/or complex data sets and may assist in generating hierarchies of clusters/groups. Alternatives exist, but may be less suited to such data sets. For instance, a common alternative clustering approach is K-means clustering, where it is required to input a priori the number of clusters. This, by definition, is generally unknown, particularly in a complex and heterogenous data set, e.g. of the type which may be encountered in medical research. Further, it may exclude processing to establish hierarchies of clusters.

In some embodiments, the baseline configuration is one of an assumption that each participant in the medical trial is in its own cluster and an assumption that each participant in the medical trial is in the same cluster.

In some embodiments, the computation of the statistical clusters comprises additionally using the algorithm that seeks to maximise the function to model a different baseline clustering configuration as a starting point and making random sequential adjustments to the configuration such that in each case the clusters are redefined so that one participant at a time is no longer in one cluster and is instead in another, analysing the effect of each adjustment on the function and where the function is larger, retaining the relevant adjustment and where the function is smaller, reversing and the relevant adjustment. This process (i.e. random sequential adjustments which are retained or reversed as appropriate) may be continued until substantially no further increases in the function are achieved. In combination, the computation starting from different baseline clustering configurations increases the likelihood of the reaching an accurate result. The likelihood of reaching an incorrect cluster solution which represents a local (but not global) maximum value for the function is reduced in this way. The approach may therefore mitigate the possibility of one of the computations becoming ‘stuck’ in a local maximum. If the two computations give different solutions in terms of the function value, the statistical cluster(s) corresponding to the higher function value may be used. This approach therefore produces further improvements given the randomness of the starting point and the randomness of the adjustments made.

In some embodiments, the different baseline configuration is the other of an assumption that each participant in the medical trial is its own cluster and an assumption that each participant in the medical trial is in the same cluster.

If the function determined at the end of the computation of the statistical clusters does not exceed a given threshold, it may be determined that there are no statistically significant clusters present. The threshold may be substantially zero.

In some embodiments, the medical trial data corresponds to only partial completion of the medical trial in that the medical trial participants constitute existing medical trial participants which are only a proportion of medical trial participants ultimately intended to form part of the medical trial, the medical trial data for the full medical trial ultimately being intended to additionally comprise medical trial data of further medical trial participants. By way of example, it may be that it is intended that the medical trial should collect medical trial data for 200 trial participants and that to date medical trial data has been collected for only 100 medical trial participants (i.e. existing medical trial participants). The medical trial would therefore be designed to include 100 further medical trial participants.

In some embodiments, the method comprises a computer implemented projection to extrapolate participant medical impact data known for the existing medical trial participants to account for at least one of the further medical trial participants and where the medical trial conclusion comprises a prediction dependent on the projection. As will be appreciated, the projecting may be performed with respect to one, some or all of the further medical trial participants. Especially when using the method, it may be that it is possible to reasonably predict outcomes e.g. efficacy, side effects, comparisons between treatments, comparisons between a treatment and a placebo or groups (which may be linked by traits) of further medical trial participants. Such predictions may mean that it is unnecessary to complete the medical trial or at least that additional insights can be gained before completion of the medical trial. This may, for example, allow for informed outcomes to be applied to the participants of the trial and/or patients generally more rapidly and/or with increased accuracy. In short, reality may be more accurately and completely simulated than is achievable with reference to the participant medical impact data known for the existing medical trial participants alone. As will be appreciated, the participant medical impact data known for the existing medical trial participants could be of the first type of participant medical impact, the second type of participant medical impact or another type of participant medical impact for which medical trial data has been collected but not completed. By making a prediction for a type of participant medical impact for which data has already been collected by the trial, this data may be used to assist in performing the projection and generating the prediction.

In some embodiments, the extrapolation is performed in accordance with a prevalence (e.g. where the data is binary e.g. death or hospitalisation) or continuous distribution (e.g. where the data is continuous e.g. extent of blood pressure change or tumour size reduction) of the corresponding participant medical impact among the existing medical trial participants. In this way, the projection may be faithful to the randomness of possible further trial participants whilst using the outcome information already acquired from the existing medical trial participants to guide the projection.

In some embodiments, the projection comprises performing a simulation instance realisation where an imaginary participant medical impact is generated for each of at least one of the further medical trial participants, the imaginary participant medical impacts being selected, in each case, at random and where the probability of any given imaginary participant medical impact arising in the random generation is consistent with the probability of the corresponding participant medical impact arising as determined by the prevalence or continuous distribution. This may be preferable to alternative projection techniques (e.g. the Student's t-test) which are predicated on additional assumptions (e.g. that the participants' data would fall substantially on a normal distribution—which may not be the case e.g. in the context of a medical trial). The recited approach preserves greater scope for randomness and heterogeneity in a system (essentially by making fewer assumptions).

In some embodiments, the projection comprises performing multiple instances of the simulation instance realisation and combining the imaginary participant medical impacts thereby generated for each simulation instance realisation with the participant medical impacts of the existing medical trial participants, wherein the mean of all such combinations is the prediction. Consequently, the prediction is a prediction of participant medical impacts for the medical trial when completed. This may allow for an improved quality of prediction. It may be that the method predicts success or failure of the medical trial in accordance with the prediction. It may be that the standard deviation between all the combinations indicates the confidence of the prediction.

In some embodiments, the projection comprises performing multiple instances of the simulation instance realisation for at least two arms of the medical trial separately and for each arm combining the imaginary participant medical impacts thereby generated for each simulation instance realisation with the participant medical impacts of the existing medical trial participants in the respective arm, wherein the prediction comprises a mean for each arm, each mean corresponding to the mean of all such combinations for the relevant arm. Consequently, the prediction is a prediction of participant medical impacts for each arm of the medical trial when completed. The prediction may additionally or alternatively comprise a comparison between each of the means. In this manner a prediction may be generated as to the difference between, for instance, two or more different treatments or a treatment and a placebo, where the medical trial for each arm is incomplete. The different arms of the trial might for example relate to different treatments (including treatment and no treatment i.e. placebo) or different dosages.

In some embodiments, the projection is performed in respect of existing medical trial participants and further medical trial participants having a particular common participant trait or group of traits. In this manner, a prediction as regards medical trial outcome that is specific to individuals having particular characteristics may be generated.

In some embodiments, the continuous distribution is generated by interpolating an empirical distribution collating the medical trial data collected for the corresponding participant medical impact among the existing medical trial participants. The interpolation may for instance be performed by kernel density estimation. This may allow the use of more accurate statistical tests and analyses to be performed on the medical trial data. By using continuous distributions generated by the kernel estimator procedure, it is possible to use statistical comparison tests that do not make assumptions about the distribution of the empirical distribution (e.g. that it is Gaussian). This may give greater accuracy in terms of predictions arising. The empirical distribution may for instance be a histogram.

In some embodiments, kernel density estimation interpolating is performed in accordance with the equation:

$${\hat{f}}_{h} ( x ) = \frac{1}{n} {\sum }_{i = 1}^{n} {K}_{h} ( x - {x}_{i} ) = \frac{1}{nh} {\sum }_{i = 1}^{n} K ( \frac{x - {x}_{i}}{h} ) \qquad Equation 3$$

$$Where :$$

$$K ( y ) = \frac{3}{4} ( 1 - {y}^{2} ) {1}_{\{ \vert y \vert < 1 \}} \qquad Equation 4$$

Where K is a kernel function, x and x; represent the mean and individual data entries for participant medical impact respectively, n is the number of such data entries, f is the inferred kernel and y is an independent variable (the value of the frequency in the kernel density estimation). The expression of Equation 4 minimises the least-squares error of the resulting kernel against the empirical data.

This approach may be well suited to the data sets which are likely to be encountered e.g. non-heterogeneous data sets, because it is not premised on an assumption of a normal (or indeed any specific) distribution. In general, given the highly heterogenous and complex nature of medical trial and other real-world data sets, it is not desirable to assume any specific form for the underlying distribution.

In some embodiments, the method comprises collecting the medical trial data of the medical trial participants.

In some embodiments, the method comprises at least one of:
i) Treating the medical trial participants in dependence on the medical trial conclusion;
ii) Treating patients in dependence on the medical trial conclusion;
iii) Designing another medical trial in dependence on the medical trial conclusion;
iv) Adapting the medical trial in dependence on the medical trial conclusion;
v) Halting the medical trial before its planned conclusion in dependence on the medical trial conclusion;
vi) Making a determination on the success or failure of the medical trial in dependence on the medical trial conclusion;
vii) Labelling, optionally automatically, a medication for treatment of one or more patients in dependence on the medical trial conclusion.

Consequently, the enhanced understanding afforded by the method in terms of the performance of the participants in the medical trial may be acted upon. To give an example of each of the options mentioned:
a) Regarding i), it could be for instance that participants in an identified group are treated (or are not treated) in a similar manner, in accordance with the effectiveness/side effects shown or predicted to be characteristic of the group. Further, participants not in that discovered group may be treated (or not treated) in a manner different to participants in the group.
b) Regarding ii), it could be that individuals beyond the trial participants are recognised as having traits in common with a group identified by the method and are correspondingly treated (or not treated) in a manner determined for such individuals on the basis of that commonality and the demonstrated or predicted characteristics of that group in terms of medical impacts (e.g. effectiveness/side effects).
c) Regarding iii), it may be that identifying one or more groups which behave or are predicted to behave in a particular manner in terms of medical impact, can inform the design of another (e.g. future) medical trial to increase the likelihood of that other medical trial giving useful/decisive results. For instance, a trial may be designed with a particular treatment or dosage that is more likely to be effective and/or trial participants may be recruited having particular traits which the medical trial conclusion suggests are indicators of likely effectiveness/likely side effect levels.
d) Regarding iv), it may be that the medical trial is yet to be completed and can be usefully adapted in dependence on the medical trial conclusion. For instance, it may be that based on apparent effectiveness/side effects associated with an identified group, it is preferable to recruit further participants to the trial in accordance with traits in common with or not in common with that group.
e) Regarding v), it may be appropriate to halt the medical trial before its completion (e.g. in accordance with the identifying of a group and/or a prediction concerning medical impact), because the medical impact for this group and/or the relative size of the group may suggest that to continue the medical trial in its existing form is likely not to be worthwhile, futile and/or dangerous.
f) Regarding vi), it may be possible to pre-empt the completion of the trial (e.g. in dependence on an identified group and/or a prediction concerning medical impact) e.g. because medical impact for this group and/or the relative size of the group may point strongly to a particular outcome for the medical trial which can then be assumed.

In some embodiments, the method for generating a medical trial conclusion is repeated where additional medical trial data is collected. As will be appreciated, the additional medical trial data may correspond to additional data for one or more existing medical trial participants and/or data for a further participant or participants. The quality of the medical trial conclusion generated may be enhanced where additional medical trial data is used.

According to an aspect of the invention there is provided a computer program that, when read by a computer, causes performance of the method as described above.

According to an aspect of the invention there is provided a non-transitory computer readable storage medium comprising computer readable instructions that, when read by a computer, cause performance of the method as described above.

According to an aspect of the invention there is provided a signal comprising computer readable instructions that, when read by a computer, cause performance of the method as described above.

According to an aspect of the invention there is provided a medical trial conclusion generating system optionally arranged to generate a medical trial conclusion optionally with respect to at least some participants in a medical trial, where medical trial data of the trial participants has optionally been collected and the medical trial data collected optionally comprises a data set pertaining to a first type of participant medical impact, the system optionally comprising:
an optional input means optionally arranged to receive the data set pertaining to the first type of participant medical impact;
an optional processing means optionally arranged to:
analyse the collected medical trial data of the trial participants optionally to identify a first participant medical impact group optionally by performing statistical clustering analysis for the participants optionally in terms of participant medical impact optionally as determined in accordance with the data set pertaining to the first type of participant medical impact, the first participant medical impact group optionally corresponding to a statistical cluster so determined; and
an optional output means optionally arranged to output a medical trial conclusion optionally comprising an indication of the first participant medical impact group.

As will be appreciated further features described above with respect to other aspects are also applicable to this aspect mutatis mutandis. Thus, purely by way of example, the medical trial data may comprise at least one data set each pertaining to a respective type of participant trait, which may be received by the input means. Further, the processing means may be arranged to analyse the collected medical trial data of the medical trial participants to identify one or more first participant traits represented within the first participant medical impact group, as determined in accordance with the at least one data set each pertaining to a respective type of participant trait. Further, the medical trial conclusion which is output by the output means may comprise an indication of at least one of the first participant traits and an indication that individuals having a trait or traits consistent with one or more of the first participant traits are more likely to experience medical impact associated with the first participant medical impact group.

According to an aspect of the invention, there is provided a non-transitory computer readable storage medium optionally having stored thereon a data structure which optionally identifies a first participant medical impact group from among medical trial participants, where medical trial data of the medical trial participants has optionally been collected and the medical trial data collected optionally comprises a data set pertaining to a first type of participant medical impact, the identification of the first participant medical impact group optionally being determined in accordance with a method optionally comprising:
optional computer implemented analysis of the collected medical trial data optionally of the medical trial participants optionally to identify the first participant medical impact group optionally by performing statistical clustering analysis for the medical trial participants optionally in terms of participant medical impact optionally as determined in accordance with the data set pertaining to the first type of participant medical impact, the first participant medical impact group optionally corresponding to a statistical cluster so determined.

As will be appreciated further features described above with respect to other aspects are also applicable to this aspect, mutatis mutandis. Thus, purely by way of example, the medical trial data may comprise at least one data set each pertaining to a respective type of participant trait. Further, the data structure may indicate at least one first participant trait and indicate that individuals having a trait or traits consistent with one or more of the first participant traits are more likely to experience medical impact associated with the first participant medical impact group, the at least one first participant traits being determined in accordance with a method comprising: analysing the collected medical trial data of the medical trial participants to identify one or more first participant traits represented within the first participant medical impact group, as determined in accordance with the at least one data set each pertaining to a respective type of participant trait.

According to an aspect of the invention there is provided a medical trial analysis system optionally comprising:
optionally a data gathering node optionally comprising collecting medical trial data optionally of trial participants in a medical trial, the medical trial data collected optionally comprising a data set optionally pertaining to a first type of participant medical impact and optionally at least one data set each pertaining to a respective type of participant trait;
optionally a computer implemented analysis node comprising:
   optional analysing of the collected medical trial data of the trial participants optionally to identify a first participant medical impact group optionally by performing statistical clustering analysis for the medical trial participants optionally in terms of participant medical impact optionally as determined in accordance with the data set pertaining to the first type of participant medical impact, the first participant medical impact group optionally corresponding to a statistical cluster so determined; and
   optionally analysing the collected medical trial data of the medical trial participants optionally to identify one or more first participant traits represented within the first participant medical impact group, optionally as determined in accordance with the at least one data set each pertaining to a respective type of participant trait; and
an optional implementation node comprising at least one of:
   optionally treating the medical trial participants optionally in dependence on the analysis of the computer implemented analysis node;
   optionally treating patients optionally in dependence on the analysis of the computer implemented analysis node;
   optionally designing another medical trial optionally in dependence on the analysis of the computer implemented analysis node;
   optionally adapting the medical trial optionally in dependence on the analysis of the computer implemented analysis node;
   optionally halting the medical trial before its planned conclusion optionally in dependence on the analysis of the computer implemented analysis node;
   optionally making a determination on the success or failure of the medical trial optionally in dependence on the analysis of the computer implemented analysis node;
optionally labelling, optionally automatically, a medication optionally for treatment of one or more patients optionally in dependence on the analysis of the computer implemented analysis node.

As will be appreciated further features described above with respect to other aspects are also applicable to this aspect, mutatis mutandis.

According to an aspect of the invention, there is provided a method for processing data, the data optionally comprising a data set pertaining to a first type of impact on entities, the method optionally comprising:
optional computer implemented analysis of the data optionally to identify a first entity impact group optionally by performing statistical clustering for the entities in terms of the first type of impact optionally as determined in accordance with the data set pertaining to the first type of impact on the entities; and
optionally generating a conclusion comprising an indication of the first entity impact group.

As will be appreciated, further features described above with respect to other aspects are also applicable to this aspect, mutatis mutandis. Thus, purely by way of example, the data may comprise at least one data set each pertaining to a respective type of contextual information for the entities. Further, the method may comprise analysing the collected data of the entities to identify one or more first contexts represented within the first entity impact group, as determined in accordance with the at least one data set each pertaining to a respective type of contextual information. Further, the conclusion may comprise an indication of at least one of the first contexts and an indication that entities having a context or contexts consistent with one or more of the first contexts are more likely to experience impact associated with the first entity impact group.

Examples of entities include pieces of equipment, companies, people, system nodes etc. Respective examples of a data set would include equipment performance data, company share price data, participant medical impact data or system node location. The approach may be significant because, in general, the majority (e.g. 90% or more) of the eigenvalues and associated eigenvectors of a raw correlation matrix may be associated with noise, and therefore the bulk of the raw correlation matrix entries may be indistinguishable from pure noise. Consequently, mitigating or reducing the effect of these eigenvalues may be significant in terms of clustering accuracy.

According to an aspect of the invention there is provided a data processing method applied to a data set optionally comprising time series data optionally pertaining to entities, the method optionally comprising:
optionally calculating a distance metric optionally between each of the entities and each of the other entities, each distance metric optionally indicating a relative degree of similarity of the respective pair of entities, the calculating of the distance metrics optionally comprising:
   optionally generating a first eigenspectrum of a correlation matrix optionally comprising correlation coefficients between the respective pairs of the entities optionally as determined in accordance with the data set;
   optionally generating a second eigenspectrum of a random matrix optionally having the same dimensions as the correlation matrix;
   optionally removing eigenvalues of the second eigenspectrum from the first eigenspectrum;
   optionally generating a final matrix using the remaining eigenvalues and optionally using these as the distance metrics.

In some embodiments, the data set comprises medical trial data and the entities are participants in the medical trial.

The random matrix may be selected to also have the same general mathematical properties (e.g. same size, symmetric and/or being positive semi-definite) as the correlation matrix.

In some embodiments, calculating the correlation coefficient between respective pairs of entities is performed in accordance with equation 1.

In some embodiments, the method comprises determining statistical clusters among the entities in accordance with the calculated distance metrics.

In some embodiments, determination of the statistical clusters comprises using an algorithm that, given the distance metrics, seeks to maximise a function which indicates the degree of assortative grouping, modelling a baseline clustering configuration as a starting point and making random sequential adjustments to the configuration such that in each case the clusters are redefined so that one entity at a time is no longer in one cluster and is instead in another, analysing the effect of each adjustment on the function and where the function is larger, retaining the relevant adjustment and where the function is smaller, reversing the relevant adjustment. This process (i.e. random sequential adjustments which are retained or reversed as appropriate) may be continued until substantially no further increases in the function are achieved. Thus, where an adjustment causes distant data points to be defined as being in the same cluster, the function would tend to be smaller and so the adjustment would tend to be reversed and vice versa. The method may therefore divide entities into groups so as to increase internal group connectedness relative to connectedness between groups. This approach may be well suited to large data sets and/or diverse data set types (as may occur in the case of a medical trial). Specifically, the methodology allows for efficient clustering analysis despite it being difficult/inappropriate to make starting assumptions concerning the clustering of the data given its potential diversity of types. It does this by allowing for initial randomness in the baseline whilst ensuring a relatively rapid convergence on a solution which will approximate an optimal solution. The approach therefore gives advantages in terms of reducing processing and time required. Additionally, the approach may offer improved results by comparison with simply performing correlation analysis on the distance metrics calculated, which would introduce global noise through the mathematical operations inherent in the computation of correlations and not reflecting signals or structure in the data itself.

In some embodiments, the function is as per equation 2.

Such a function may assist in more accurately identifying clusters in a heterogenous and/or complex data sets and may assist in generating hierarchies of clusters/groups. Alternatives exist, but may be less suited to such data sets. For instance, a common alternative clustering approach is K-means clustering, where it is required to input a priori the number of clusters. This, by definition, is generally unknown, particularly in a complex and heterogenous data set, e.g. of the type which may be encountered in medical research. Further, it may exclude processing to establish hierarchies of clusters.

In some embodiments, the baseline configuration is one of an assumption that each entity is in its own cluster and an assumption that each entity is in the same cluster.

In some embodiments, the computation of the statistical clusters comprises additionally using the algorithm that seeks to maximise the function to model a different baseline clustering configuration as a starting point and making random sequential adjustments to the configuration such that in each case the clusters are redefined so that one entity at a time is no longer in one cluster and is instead in another, analysing the effect of each adjustment on the function and where the function is larger, retaining the relevant adjustment and where the function is smaller, reversing the relevant adjustment. This process (i.e. random sequential adjustments which are retained or reversed as appropriate) may be continued until substantially no further increases in the function are achieved. In combination, the computation starting from different baseline clustering configurations increases the likelihood of reaching an accurate result. The likelihood of reaching an incorrect cluster solution which represents a local (but not global) maximum value for the function is reduced in this way. The approach may therefore mitigate the possibility of one of the computations becoming ‘stuck’ in a local maximum. If the two computations give different solutions in terms of the function value, the statistical clusters corresponding to the higher function value may be used. This approach therefore produces further improvements given the randomness of the starting point and the randomness of the adjustments made.

In some embodiments, the different baseline configuration is the other of an assumption that each entity is its own cluster and an assumption that each entity is in the same cluster.

If the function determined at the end of the computation of the statistical clusters does not exceed a given threshold, it may be determined that there are no statistically significant clusters present. The threshold may be substantially zero.

In some embodiments, the method comprises collecting the data of the data set.

According to an aspect of the invention there is provided a computer program that, when read by a computer, causes performance of the method as described above.

According to an aspect of the invention there is provided a non-transitory computer readable storage medium comprising computer readable instructions that, when read by a computer, cause performance of the method as described above.

According to an aspect of the invention there is provided a signal comprising computer readable instructions that, when read by a computer, cause performance of the method as described above.

According to an aspect of the invention there is provided a data processing system optionally arranged to process a data set optionally comprising time series data pertaining to entities, the system optionally comprising:
an optional input means optionally arranged to receive the data set pertaining to the entities;
an optional processing means optionally arranged to:
   optionally calculate a distance metric optionally between each of the entities and each of the other entities, each distance metric optionally indicating a relative degree of similarity of the respective pair of entities, the calculating of the distance metrics optionally comprising:
      optionally generating a first eigenspectrum of a correlation matrix comprising correlation coefficients between the respective pairs of the entities optionally as determined in accordance with the data set;
      optionally generating a second eigenspectrum of a random matrix optionally having the same dimensions as the correlation matrix;
      optionally removing eigenvalues of the second eigenspectrum from the first eigenspectrum; and
      optionally generating a final matrix using the remaining eigenvalues; and
an optional output means optionally arranged to output the remaining eigenvalues as the distance metrics.

As will be appreciated further features described above with respect to other aspects are also applicable to this aspect mutatis mutandis.

According to an aspect of the invention, there is provided a non-transitory computer readable storage medium optionally having stored thereon distance metrics between each of a plurality of entities and each of the other of the plurality of entities, each distance metric optionally indicating a relative degree of similarity of the respective pair of entities, the distance metrics optionally being calculated in accordance with a method comprising:
optional computer implemented generating of a first eigenspectrum of a correlation matrix optionally comprising correlation coefficients between the respective pairs of the entities optionally as determined in accordance with a data set comprising time series data pertaining to the entities;
optional computer implemented generating of a second eigenspectrum of a random matrix optionally having the same dimensions as the correlation matrix;
optional computer implemented removing of eigenvalues of the second eigenspectrum from the first eigenspectrum;
optional computer implemented generating of a final matrix using the remaining eigenvalues and optionally using these as the distance metrics.

As will be appreciated, further features described above with respect to other aspects are also applicable to this aspect, mutatis mutandis.

According to an aspect of the invention there is provided a data processing system optionally comprising:
optionally a data gathering node optionally comprising collecting a data set optionally comprising time series data pertaining to entities;
optionally a computer implemented analysis node optionally comprising:
   optionally calculating a distance metric between each of the entities and each of the other entities, each distance metric optionally indicating a relative degree of similarity of the respective pair of entities, the calculating of the distance metrics optionally comprising:
      optionally generating a first eigenspectrum of a correlation matrix optionally comprising correlation coefficients between the respective pairs of the entities optionally as determined in accordance with the data set;
      optionally generating a second eigenspectrum of a random matrix optionally having the same dimensions as the correlation matrix;
      optionally removing eigenvalues of the second eigenspectrum from the first eigenspectrum;
      optionally generating a final matrix using the remaining eigenvalues,
optionally an implementation node optionally comprising using the remaining eigenvalues as the distance metrics and optionally statistically clustering the entities in accordance with the distance metrics and optionally managing the entities in accordance with the respective statistical clusters to which they belong.

As will be appreciated, further features described above with respect to other aspects are also applicable to this aspect, mutatis mutandis.

According to an aspect of the invention there is provided a computer implemented method of projecting further data values for a partially complete data acquisition process optionally pertaining to an entity or group of entities, the partially complete data acquisition process optionally having acquired existing data values of a type and which are collated in an empirical distribution, the method optionally comprising:
optionally generating a continuous distribution by interpolating the empirical distribution; and
optionally extrapolating in accordance with the continuous distribution optionally to generate a simulation instance realisation optionally comprising imaginary data values optionally by:
   optionally generating random further data values of the type, where the probability of any given value of the further data arising in the random generation is optionally consistent with the probability of the corresponding data value arising as determined by the continuous distribution,
where optionally the imaginary data values are optionally the projected further data values or optionally the projected further data values are determined in dependence on the imaginary data values.

In this way, the projection may be faithful to the randomness of possible further data values whilst using the existing data acquired to guide the projection. It may be preferable to alternative projection techniques (e.g. the Student's t-test) which are predicated on additional assumptions (e.g. that the participants would fall substantially on a normal distribution—which may not be the case e.g. in the context of a medical trial). The recited approach preserves greater scope for randomness in a system (essentially by making fewer assumptions). By projecting further data values, a prediction of an outcome in terms of likely future data values may be achieved. In this way it may prove unnecessary to complete or perform further real-world data acquisition and/or an advance indication of the likely outcome may be provided (which may for instance allow for pre-emptive action to be taken). The empirical distribution may for instance be a histogram.

In some embodiments, the partially complete data acquisition process pertains to a medical trial and optionally the existing data values pertain to a type of participant medical impact.

As will be appreciated, the number of further data values projected may be pre-determined e.g. in order to fulfil a pre-determined requirement for a total number of data values or a given ratio of existing data values to further data vales.

In some embodiments, the projection comprises performing multiple instances of the simulation instance realisation and combining the imaginary data values thereby generated for each simulation instance realisation with the existing data values, wherein the mean values of all such combinations are the projected further data values. This may allow for an improved quality of projection in terms of the projected further data values serving as a prediction in the event that further data acquisition occurred. It may be that the standard deviation between all the combinations indicates the confidence of the projection as a prediction.

The interpolation may for instance be performed by kernel density estimation. By using continuous distributions generated by the kernel estimator procedure, it is possible to use statistical comparison tests that do not make assumptions about the distribution of the empirical distribution (e.g. that it is Gaussian or indeed any specific assumed distribution). This may give greater accuracy in terms of the projection arising.

In some embodiments, kernel density estimation interpolating is performed in accordance with equations 3 and 4.

This approach may be well suited to some data sets encountered e.g. non-heterogeneous, because it is not premised on an assumption of a normal (or other specific) distribution.

According to an aspect of the invention there is provided a computer program that, when read by a computer, causes performance of the method as described above.

According to an aspect of the invention there is provided a non-transitory computer readable storage medium comprising computer readable instructions that, when read by a computer, cause performance of the method as described above.

According to an aspect of the invention there is provided a signal comprising computer readable instructions that, when read by a computer, cause performance of the method as described above.

According to an aspect of the invention there is provided a data value projection system optionally arranged to project further data values for a partially complete data acquisition process optionally pertaining to an entity or group of entities, the system optionally comprising:
optionally an input means optionally arranged to receive acquired existing data values of a type and which are collated in an empirical distribution;
optionally a processing means optionally arranged to:
   optionally generate a continuous distribution optionally by interpolating the empirical distribution; and
   optionally extrapolating in accordance with the continuous distribution optionally to generate a simulation instance realisation comprising imaginary data values by:
      optionally generating random further data values of the type, where the probability of any given value of the further data arising in the random generation is optionally consistent with the probability of the corresponding data value arising optionally as determined by the continuous distribution; and
optionally an output means optionally arranged to output the imaginary data values as the projected further data values or optionally output the projected further data values determined in dependence on the imaginary data values.

As will be appreciated further features described above with respect to other aspects are also applicable to this aspect, mutatis mutandis.

According to an aspect of the invention, there is provided a non-transitory computer readable storage medium optionally having stored thereon projected further data values for a partially complete data acquisition process optionally pertaining to an entity or group of entities, the partially complete data acquisition process optionally having acquired existing data values of a type and which are collated in an empirical distribution, the projected further data values optionally being calculated in accordance with a method comprising:
optional computer implemented generating of a continuous distribution optionally by interpolating the empirical distribution; and
optional computer implemented extrapolating in accordance with the continuous distribution optionally to generate a simulation instance realisation comprising imaginary data values optionally by:
   optionally generating random further data values of the type, where the probability of any given value of the further data arising in the random generation is optionally consistent with the probability of the corresponding data value arising as determined by the continuous distribution,
where optionally the imaginary data values are optionally the projected further data values or the projected further data values are optionally determined in dependence on the imaginary data values.

As will be appreciated further features described above with respect to other aspects are also applicable to this aspect, mutatis mutandis.

According to an aspect of the invention there is provided a data value projection system optionally arranged to project further data values for a partially complete data acquisition process optionally pertaining to an entity or group of entities comprising:
optionally a data gathering node optionally comprising collecting acquired existing data values of a type and which are optionally collated in an empirical distribution;
optionally a computer implemented analysis node optionally comprising:
   optionally generating a continuous distribution by interpolating the empirical distribution; and
   optionally extrapolating in accordance with the continuous distribution to optionally generate a simulation instance realisation optionally comprising imaginary data values optionally by:
      optionally generating random further data values of the type, where the probability of any given value of the further data arising in the random generation is optionally consistent with the probability of the corresponding data value optionally arising as determined by the continuous distribution, where optionally the imaginary data values are the projected further data values or optionally the projected further data values are determined in dependence on the imaginary data values;
optionally an implementation node optionally comprising using the projected further data values as a proxy for data values that would be acquired by completing the data acquisition process and optionally aborting completion of the data acquisition process.

As will be appreciated further features described above with respect to other aspects are also applicable to this aspect, mutatis mutandis.

Any controller or controllers described herein may suitably comprise a control unit or computational device having one or more electronic processors. Thus the system may comprise a single control unit or electronic controller or alternatively different functions of the controller may be embodied in, or hosted in, different control units or controllers. As used herein the term “controller” or “control unit” will be understood to include both a single control unit or controller and a plurality of control units or controllers collectively operating to provide any stated control functionality. To configure a controller, a suitable set of instructions may be provided which, when executed, cause said control unit or computational device to implement the control techniques specified herein. The set of instructions may suitably be embedded in said one or more electronic processors. Alternatively, the set of instructions may be provided as software saved on one or more memory devices associated with said controller to be executed on said computational device. A first controller may be implemented in software run on one or more processors. One or more other controllers may be implemented in software run on one or more processors, optionally the same one or more processors as the first controller. Other suitable arrangements may also be used.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim, although not originally claimed in that manner.

### BRIEF DESCRIPTION OF THE DRAWINGS

One or more embodiments of the invention will now be described by way of example only, with reference to the accompanying drawings, in which:

FIG. 1 shows a method for generating a medical trial conclusion according to an embodiment of the invention;

FIG. 2 shows a method for computer implemented projection to generate a prediction for an incomplete medical trial;

FIG. 3 shows a medical trial analysis system according to an embodiment of the invention; and

FIG. 4 shows a medical trial conclusion generating system according to an embodiment of the invention.

### DETAILED DESCRIPTION

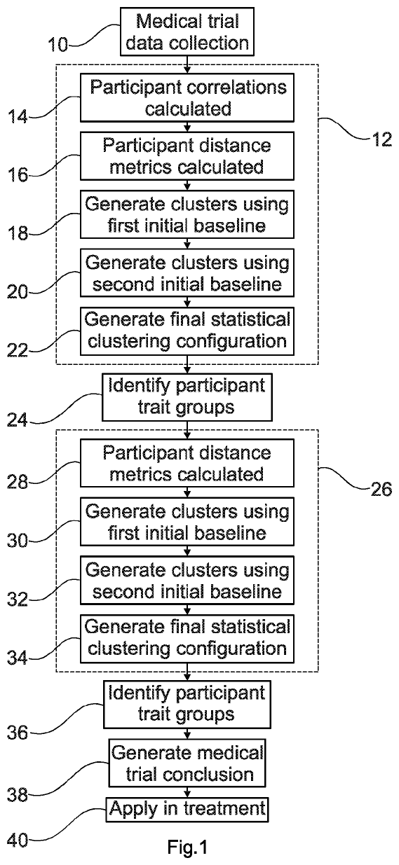

Referring first to FIG. 1, a method for processing data in order to generate a conclusion is provided in outline. By way of illustration only, the method is applied to a medical trial in a manner such that it is a method for generating a medical trial conclusion.

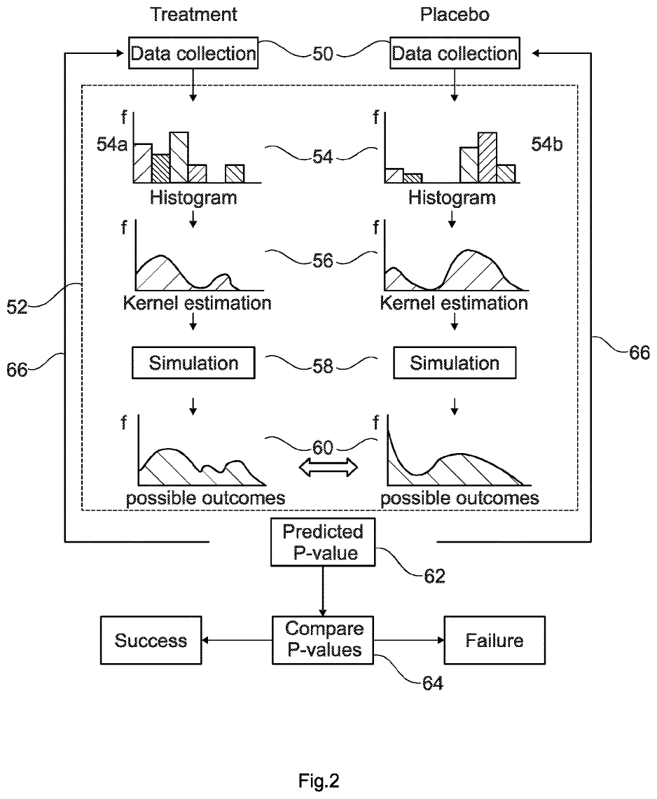

In an example scenario, the medical trial is to investigate the performance of a cancer drug when administered in two different dosage regimes (regime 1 and regime 2) corresponding to respective first and second arms of the medical trial. The medical trial is complete in that all data has been collected for all intended participants. Nonetheless, this is not necessary, as the method can be conducted with only partial completion of the medical trial using data collected to date or on a combination of data collected to date and projected data as discussed further below with respect to FIG. 2.

Within the medical trial data collected is data collected pertaining to a first type of impact on entities, in this case data pertaining to a first type of participant medical impact. The first type of participant medical impact may be a type in the sense that it concerns a particular indicator, consistently used across the participants, regarding a particular medical characteristic or status which may arise because of and/or be impacted by the medical trial (e.g. a symptom, side effect, statistic or condition affecting part or all of the body etc). As will be appreciated, the data may for example indicate that for a particular participant: the medical impact is or is not present, where appropriate (e.g. continuous type) the degree to which the medical impact is present and/or falls into a particular category etc. Together, the medical trial data collected for the first type across all participants is a data set pertaining to the first type of participant medical impact. In the present scenario, the first type of participant medical impact is tumour size as recorded at intervals over time and as quantified in width or volume.

Also within the medical trial data collected, is data collected pertaining to a second type of impact on entities, in this case data pertaining to a second type of participant medical impact. In the present scenario, the second type of participant medical impact is extent of neutropenia at one month after treatment as quantified in absolute neutrophil count (ANC). Together, the medical trial data collected for the second type across all participants is a data set pertaining to the second type of participant medical impact.

Also within the medical trial data collected, is data pertaining to a type of contextual information of the entities, in this case data pertaining to a type of participant trait. The type of participant trait may be a type in the sense that it concerns a particular indicator, consistently used across the participants, as regards a particular feature of the participant and/or their circumstances (e.g. height, weight, age, ethnicity, genetic characteristic, disease stage arm of the medical trial etc). As will be appreciated, the data may for example indicate that for a particular participant: the type of participant trait is or is not present, where appropriate (e.g. continuous type) the degree to which the type of participant trait is present, and/or falls into a particular category etc. Together, the medical trial data collected for the type of participant trait across all participants is a data set pertaining to the type of participant trait. In the present scenario, data is collected pertaining to multiple types of participant trait: in this case, gender, body mass index (BMI) and BRAF gene mutation presence.

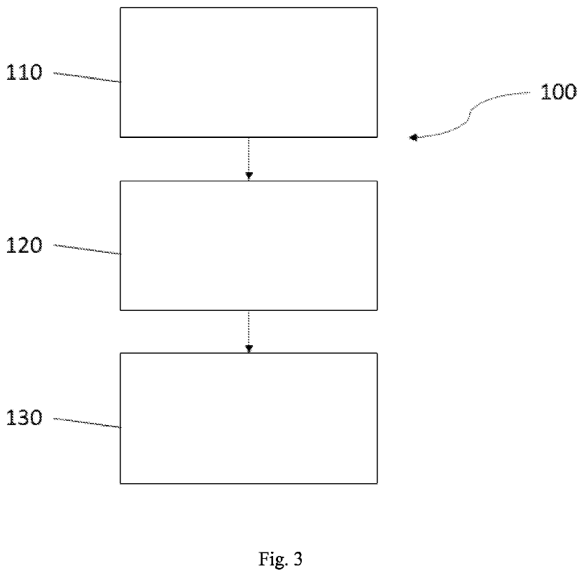

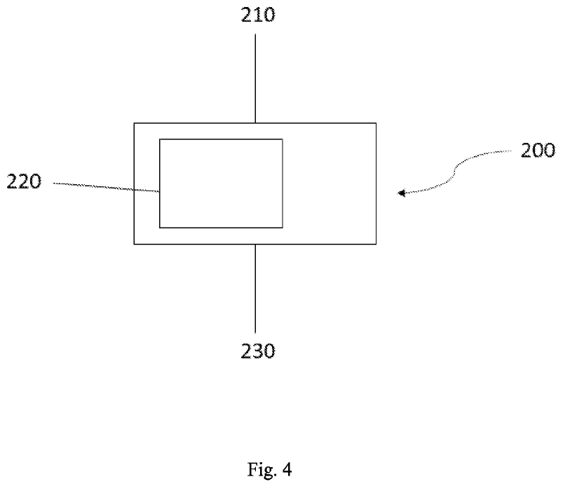

The medical trial data is collected at step 10 of FIG. 1, this may be considered a data gathering node 110 of a medical trial analysis system 100 (see FIG. 3) and/or may be input to a medical trial conclusion generating system 200 via an input means 210 thereof (see FIG. 4).

The group of steps 12 correspond to a clustering process performed with a view to identifying one or more entity impact groups, in this case participant medical impact groups, in the arm for dosage regime 1. To achieve this, the tumour size data for the medical trial participants in the dosage regime 1 arm is analysed to seek statistical clusters of participants according to that data.

The clustering process is performed in broad terms by calculating a distance metric between each of the medical trial participants in the relevant arm of the medical trial and each of the other medical trial participants in that arm, each distance metric indicating a relative degree of similarity of the respective pair of medical trial participants in terms of the tumour size data. An algorithm is then used to iteratively define statistical clusters of the medical trial participants given the distance metric information.

In order to calculate each distance metric and because the tumour size data is time series data, a correlation coefficient between each medical trial participant in the relevant arm and each other medical trial participant in the relevant arm is first calculated in step 14. This indicates how similar each is to each other in terms of tumour size. In this example, the correlation coefficient (r_{xy}) is calculated in accordance with equation 1.

In step 16, using the correlations calculated, a distance metric between each medical trial participant in the relevant arm and each other medical trial participant in the relevant arm is calculated. This is achieved by generating a first eigenspectrum of a correlation matrix comprising the correlation coefficients and then generating a second eigenspectrum of a random matrix having the same dimensions and general mathematical properties as the correlation matrix. This allows for the removal of the eigenvalues of the second eigenspectrum from the first eigenspectrum by setting the eigenvalues of the second eigenspectrum to zero, which renders their associated eigenvectors also 0, and consequently they do not contribute to the distance metric. A final matrix is generated using the eigenvalues that are left:

$${C}^{( g )} ≡ {\sum }_{i : {λ}_{+} < {λ}_{i} < {λ}_{m}} {λ}_{i} \vert {v}_{i} 〉 〈 {v}_{i} \vert \qquad Equation 5$$

Where C^{(g)} is the distance metric, lambda represents the eigenvalues of the correlation matrix and v is the associated eigenvector to each eigenvalue.

The eigenvalues of the final matrix are the distance metrics.

It is noteworthy that the approach of calculating a distance metric using the eigenspectrum technique discussed can be used independently of the other steps indicated here. It may therefore be used as a stand-alone data processing method applied to a data set comprising time series data pertaining to entities.

In step 18, equation 2 is used with the distance metrics as inputs. An algorithm makes random, sequential adjustments to an initial baseline statistical clustering configuration of all the medical trial participants in the relevant arm with a view to iteratively maximising the function Q. The function Q indicates degree of assortative grouping. The initial baseline statistical clustering configuration is one where each medical trial participant in the relevant arm is in its own cluster. Each random adjustment results in redefining the statistical clusters of the medical trial participants in the relevant arm. Specifically, each adjustment is such that one medical trial participant at a time is no longer in one cluster and is instead in another. The effect of the adjustment is then analysed in terms of the effect on the function. The adjustment is retained if it resulted in the function being larger and reversed if it resulted in the function being smaller. This process is continued until substantially no further increases in the function are achieved (or are likely).

In step 20, step 18 is independently repeated, but with the initial baseline statistical clustering configuration used in step 18 replaced. In step 20, the initial baseline statistical clustering configuration is one where each medical trial participant in the relevant arm is in the same cluster.

In step 22, the final statistical clustering configuration achieved in step 18 is compared with that achieved in step 20, with the configuration giving rise to the higher function Q being selected as the statistical clustering configuration. Taking this approach increases the likelihood of clustering optimisation. For instance, it may assist in preventing settling on a statistical clustering configuration which has found only a local maximum.

For the purposes of the example scenario, it is assumed that step 22 returns a statistical clustering configuration whereby a cluster of medical trial participants are found to have had a large reduction in tumour size over time, another cluster having a small reduction in tumour size over time and another seeing an increase in tumour size over time as well as a number of non-clustered participants. In this example, the cluster of medical trial participants having a large reduction in tumour size over time is arbitrarily selected as a first participant medical impact group for further analysis, though the steps which follow could equally and/or additionally be performed with respect to other of the identified clusters.

In step 24, analysis is performed with a view to identifying one or more statistically significant entity context groups, in this case participant trait groups, represented within the first participant medical impact group. To achieve this, each of the data sets pertaining to a respective type of participant trait are analysed in turn with respect to the medical trial participants found to be in the first participant medical impact group. Therefore, in the example scenario, analysis is performed to establish whether the first participant medical impact group contains a statistically significant group of medical trial participants with respect to gender, whether it contains a statistically significant group with respect to BMI and whether it contains a statistically significant group with respect to the BRAF gene mutation being present or otherwise. Additionally, consideration can be given to whether there is a statistically significant group having a combination of traits (e.g. in this case analysis is performed to determine whether the first participant medical impact group contains a statistically significant combination of a particular gender and a particular BRAF gene mutation characteristic). In the example scenario, this analysis leads to the conclusion that the first participant medical impact group contains a statistically significant group comprising women who lack the BRAF gene mutation. This is classed as a first participant trait group.

By way of an example the method of determining statistical significance of any clusters identified (e.g. whether or not a cluster corresponds to a statistically significant participant trait group) the following approach may be taken. The cluster considered may have a ‘quality’ measure calculated over its membership, in accordance with Equation 2. Then, the cluster may be determined to be statistically significant if its quality value is larger than those for clusters of the same and/or similar size same size detected in randomised networks of the same and/or similar size as the network under analysis. Statistical significance may also be assessed in a similar manner regarding the absence of clusters if it is determined that there are no clusters.

The group of steps 26, corresponds to a clustering process performed with a view to identifying one or more entity impact sub-groups, in this case participant medical impact sub-groups, within the first participant medical impact group (large reduction in tumour size over time in the first arm of the medical trial). To achieve this, statistical clustering analysis for the medical trial participants in the first participant medical impact group is performed in terms of medical trial participant medical impact as determined in accordance with the data set pertaining to the second type of participant medical impact (in this case extent of neutropenia at one month after treatment).

The clustering process is performed in broad terms by calculating a distance metric between each of the medical trial participants in the first participant medical impact group and each of the other medical trial participants in the first participant medical impact group, each distance metric indicating a relative degree of similarity of the respective pair of medical trial participants in terms of the extent of neutropenia data. An algorithm is then used to iteratively define statistical clusters of the medical trial participants given the distance metric information.

In this case, and because the neutropenia data is not time series data, the distance metrics are simply the differences in the ANC values for the respective pairs of medical trial participants. These distance metrics are calculated in step 28.

In step 30, an adaptation of equation 2 is used with the distance metrics as inputs. The adaptation arises because the neutropenia data is not time series data (but is rather individual measurements) and so eigenspectral cleaning is not necessary. Thus, equation 2 is adapted such that the distance metrics used to calculate the function Q are the relative differences in the ANC values for the respective pairs of medical trial participants (rather than eigenvalues arising from eigenspectral cleaning).

An algorithm makes random, sequential adjustments to an initial baseline statistical clustering configuration of all the medical trial participants in the first participant medical impact group with a view to iteratively maximising the function Q. The function Q indicates degree of assortative grouping. The initial baseline statistical clustering configuration is one where each medical trial participant in the first participant medical impact group is in its own cluster. Each random adjustment results in redefining the statistical clusters of the medical trial participants within the first participant medical impact group. Specifically, each adjustment is such that one medical trial participant at a time is no longer in one cluster and is instead in another. The effect of the adjustment is then analysed in terms of the effect on the function. The adjustment is retained if it resulted in the function being larger and reversed if it resulted in the function being smaller. This process is continued until substantially no further increases in the function are achieved.

In step 32, step 30 is independently repeated, but with the initial baseline statistical clustering configuration used in step 30 replaced. In step 32, the initial baseline statistical clustering configuration is one where each medical trial participant in the first participant medical impact group is in the same cluster.

In step 34, the statistical clustering configuration achieved in step 30 is compared with that achieved in step 32, with the configuration giving rise to the higher function Q being selected as the final statistical clustering configuration.

For the purposes of the example scenario, it is assumed that step 34 returns a statistical clustering configuration whereby a cluster of medical trial participants are found to have suffered substantially no reduction in ANC and a cluster of medical trial participants have suffered significant reduction in ANC commensurate with neutropenia as well as a number of non-clustered participants. In this example, the cluster of medical trial participants having substantially no reduction in ANC is arbitrarily selected as a first participant medical impact sub-group for further analysis, though the steps which follow could equally and/or additionally be performed with respect to the other identified cluster.

In step 36, analysis is performed with a view to identifying one or more statistically significant entity context groups, in this case participant trait groups, represented within the first participant medical impact sub-group. In this case, the data set pertaining to BMI is analysed with respect to the medical trial participants found to be in the first participant medical impact sub-group. In the example scenario, this analysis leads to the conclusion that the first participant medical impact sub-group contains a statistically significant group comprising medical trial participants classed as overweight or obese on the BMI scale. This is classed as a second participant trait group.

In step 38, a medical trial conclusion is generated comprising in this example:
i) An indication that for participants receiving dosage regime 1, participants that are female and do not have a BRAF gene mutation are more likely to see a significant reduction in tumour size; and
ii) An indication that for participants receiving dosage regime 1 and seeing a significant reduction in tumour size, those that are overweight or obese are more likely to experience neutropenia.

The preceding steps after step 10 may be considered a computer implemented analysis node 120 of the medical trial analysis system 100 and/or may be performed in part or in full by a processing means 220 of the medical trial conclusion generating system 200.

The medical trial conclusion may then be stored as a data structure, for example in a non-transitory computer readable storage medium and/or may be output e.g. by an output means 230 of the medical trial conclusion generating system 200.

In step 40 the medical trial conclusions are used in treatment choices for patients, in particular selecting and using the treatment with dosage regime 1 preferentially for women without BRAF gene mutation who are not overweight/obese. As will be appreciated however, this use of the medical trial conclusion is just one possible example and numerous other possibilities exist, e.g. treating the medical trial participants in dependence on the medical trial conclusion, designing another medical trial in dependence on the medical trial conclusion, adapting the medical trial in dependence on the medical trial conclusion, halting the medical trial in dependence on the medical trial conclusion, making a determination on the success or failure of the medical trial in dependence on the medical trial conclusion and labelling, optionally automatically, a medication for treatment of one or more patients in dependence on the medical trial conclusion. Step 40 may be considered an implementation node 130 of the medical trial analysis system 100.

The scenario above is one example, but many alternatives are possible both within the context and medial trials and beyond. As will be appreciated, even in this example, further conclusions as part of the medical trial conclusion might well be generated where for instance analysis is conducted on additional of the participant medical impact groups and/or sub-groups and/or the dosage regime 2 arm. Further still, it will be appreciated that additional types of participant trait might have been collected and used in the analysis. Such modifications would however in general require only repetition of some or all of the steps mentioned above, with appropriate substitutions in terms of subject matter at issue. As will also be appreciated, particularly where multiple arms are analysed, comparisons may be made between the arms and/or conclusions adjusted and/or caveated as part of the medical trial conclusion.

Additionally, where participant medical impact groups or sub-groups are identified, analysis and/or conclusion beyond participant trait composition and groups may be undertaken and provided as part of the medical trial conclusion. For instance, heterogeneity within a given participant medical impact group or sub-group may be considered and/or analysed.

In scenarios where no participant medical impact groups are identified, this fact itself can form the medical trial conclusion and/or an additional activity leading to an additional/alternative medical trial conclusion may be undertaken (e.g. projection as discussed further with respect to FIG. 2). If no statistically significant participant trait representations/groups are identified, the medical trial conclusion may indicate participant trait composition/representations of an identified participant medical impact group or sub-group and optionally indicate these participant traits as indicators for or risk factors for the participant medical impact group or sub-group. Additionally or alternatively, the whole participant medical impact group may be considered a functional group, i.e. a group responding in a similar manner in terms of medical impact. The reasons for the similarity may be unknown, but it may nonetheless be useful to understand that there is a group of similarly responding participants and/or it may suggest further analysis, e.g. with respect to further participant traits.

Referring now to FIG. 2, an example of a method is provided, the method being for a computer implemented projection to extrapolate participant medical impact data which is known, to generate a prediction for an incomplete medical trial dependent on the projection. Where a medical trial is incomplete, this method may be used independently of, or in combination with, the method described with reference to FIG. 1.

In an example scenario, the medical trial is to investigate the performance of a blood pressure drug. The medical trial design includes two trial arms, a control group who are administered a placebo and a treated group who are administered the blood pressure drug. The medical trial is designed for a given number of participants in total and is only partially complete to date (that is, in this case, data has been collected for 60 participants of a planned 100). Consequently, the medical trial has existing medical trial participants about whom medical trial data has been collected, and theoretical further medical trial participants about whom no medical trial data has yet been collected and about whom medical trial data would conventionally be collected in order to complete the medical trial and draw conclusions. It should be appreciated that the further medical trial participants may be in whole or in part specific individuals already identified and/or unknown individuals yet to be recruited. Additionally, in the scenario, the medical trial is ongoing, such that medial trial data is being added to the existing corpus as the medical trial is commenced for new individuals (i.e. the further medical trial participants). As will be appreciated, this process leads to participants who were formally further medical trial participants becoming existing medical trial participants.

Within the medical trial data collected is data collected pertaining to a first type of impact on entities, in this case data pertaining to a type of participant medical impact. In the present scenario, the type of participant medical impact is blood pressure after two weeks of treatment. This data is collected for both control (placebo) and treatment (blood pressure drug) groups.

Also within the medical trial data collected is data pertaining to a type of contextual information for the entities, in this case data pertaining to a type of participant trait. In the present scenario, the type of participant trait is the trial arm to which the medical trial participant belongs (i.e. placebo or blood pressure drug).

In the scenario, in order for the medical trial to be successful in the sense of demonstrating that the blood pressure drug is having a statistically meaningful impact, the results must indicate a p-value of less than 0.05 as demonstrated by the medical trial data, this being a typical accepted threshold for establishing statistical significance. The purpose of the method is to form a prediction in terms of the outcome of the medical trial given this success criterion, despite the incomplete nature of the medical trial.

The method begins at step 50 with collecting the medical trial data of the existing medical trial participants for both arms of the medical trial. That is, data indicating medical trial arm and blood pressure after two weeks of administering the blood pressure drug or placebo is collected. This may be considered a data gathering node of a medical trial analysis system and/or may be input to a medical trial conclusion generating system via an input means.

In a group of steps 52, a process of extrapolating from the medical trial data for the existing medical trial participants is performed. In step 54, for each arm of the trial, the blood pressure values are collated in an empirical distribution (in this case a histogram) of number of participants vs blood pressure value (a treated histogram 54a and a placebo histogram 54b respectively). In other examples, it may be that step 54 is omitted because the blood pressure values are acquired in the form of such histograms.

In step 56, each histogram 54a, 54b is interpolated by kernel density estimation to generate a continuous distribution. The interpolation is performed in accordance with equations 3 and 4. That is, in this case x in equation 3 is the mean of all the blood pressure values in the relevant arm, xᵢ is each blood pressure value in the relevant arm and n is the number of blood pressure values in the relevant arm.

In step 58, each continuous distribution so produced is used in generating a projection by stochastically simulating multiple possible realisations for the blood pressure values of the further medical trial participants in the respective arm of the trial. Specifically, in this scenario, in each arm, an imaginary participant medical impact (in this case blood pressure value) is generated for each of the 20 further medical trial participants (40 in total, 20 assigned to each of the treatment and placebo arms) in a first instance of the simulation. Each blood pressure value simulated is selected at random, where the probability of any given blood pressure value arising in the random generation is consistent with the probability of the corresponding blood pressure value arising as determined by continuous distribution for the relevant arm. For each arm, this simulation process is repeated multiple times e.g. thousands of times, each producing a simulation instance realisation. As will be appreciated, the random element of the simulation will produce variation in the simulated blood pressure values across the simulation instance realisations. Each simulation instance realisation in the relevant arm is then combined with the known blood pressure values for the existing medical trial participants in that arm to produce multiple possible outcomes (one being represented for each arm at 60). For each arm, the mean distribution of the combination outcomes specific to that arm is a prediction for the relevant trial arm if completed and the standard deviation between the combination outcomes for each arm indicates the confidence for the respective arm.

This approach may be preferable to (for instance) empirical trend extrapolation from the medical trial data for the existing medical trial participants, given the potential variability in the medical trial data (particularly in the early stages) and it being uncertain a priori what empirical model would be best used for the medical trial data.

It is noteworthy that the approach of interpolating to generate a continuous distribution and then generating a projection by simulation discussed can be used independently of the other steps indicated here. It may therefore be used as a stand-alone method of projecting further data values for a partially complete data acquisition process pertaining to an entity or group of entities.

In step 62, a prediction for the outcome of the medical trial in terms of a predicted p-value is determined. This is determined by comparing the two mean distributions of the combinations, one for each arm, statistically. Various techniques can be used for this statistical comparison, including, but not limited to, the two-sample Kolmogorov-Smirnoff test or Mann-Whitley test.

In step 64, the predicted p-value is compared with the p-value required for success of the medical trial and a prediction as regards success or failure of the medical trial is made in consequence. This predication, as regards success or failure, constitutes a medical trial conclusion. The preceding steps after step 50 may be considered a computer implemented analysis node of the medical trial analysis system and/or may be performed in part or in full by a processing means of the medical trial conclusion generating system.

The medical trial conclusion may then be stored as a data structure, for example in a non-transitory computer readable storage medium.

This approach allows for a virtual twin medical trial that converges to the spread and specific distribution of outcomes of an equivalent real medical trial. This in turn allows for improved accuracy of trial outcome prediction, without the need for computation power required to generate sufficient realisations to essentially eliminate stochasticity. In later stages, the approach will ‘collapse’ towards the precise outcome that would be produced by completion of the real medical trial.

As additional medical trial data is acquired (i.e. as medical trial data is obtained for additional medical trial participants e.g. one or some of the further medical trial participants), the method can be repeated (represented at 66), with each repetition resulting in a refinement of the predication.

In accordance with the medical trial conclusion, mitigation or another action may be taken prior to completion of the medical trial (this may be considered an implementation node of the medical trial analysis system). In this regard, due note may be taken of the confidence of the prediction, as indicated by the standard deviations (or confidence intervals) discussed previously. It may be, for instance, that early treatment decisions are made for medical trial participants and/or other patients, adapting the design of the medical trial, designing another medical trial in dependence on the medical trial conclusion, halting the medical trial and/or using the prediction as a proxy for completing the medical trial in terms of determining its success or failure.

The approach allows for a virtual twin clinical trial that converges to the spread and specific distribution of outcomes of the real trial, allowing for improved accuracy of trial outcome prediction, without the need for computation power required to generate sufficient realisations to essentially eliminate stochasticity.

Although not indicated in the example provided here, projection may be performed in respect of existing medical trial participants and further medical trial participants having a particular common participant trait or group of traits or belonging to a particular participant medical impact group, as might for instance be determined in a similar manner to that described with respect to the example of FIG. 1.

As will be appreciated, in other scenarios, the participant medical impact data to be extrapolated will not be of a character whereby a histogram and continuous distribution is necessary/appropriate to provide probability to guide stochastic simulation. For instance, it may be that the participant medical impact data has binary values (e.g. yes/no—for instance whether hospitalisation has occurred). In such cases, the relevant steps of the method may be omitted and the simple prevalence of the different values used in generating each simulation instance realisation (i.e. the probability of any given imaginary participant medical impact value arising in the random generation) is consistent with the prevalence of that value in the participant medical impact data for the existing medical trial participants.

As will be appreciated, whilst the examples recited here have been generally provided in the context of a medical trial, this is not intended to be limiting. The principles and techniques described have application in other fields (e.g. where it may be desirable to group entities by impacts upon them and optionally context for those entities and/or where it may be desirable to predict further data in dependence on incomplete existing data).

It will be appreciated that embodiments of the present invention can be realised in the form of hardware, software or a combination of hardware and software. Any such software may be stored in the form of volatile or non-volatile storage such as, for example, a storage device like a ROM, whether erasable or rewritable or not, or in the form of memory such as, for example, RAM, memory chips, device or integrated circuits or on an optically or magnetically readable medium such as, for example, a CD, DVD, magnetic disk or magnetic tape. It will be appreciated that the storage devices and storage media are embodiments of machine-readable storage that are suitable for storing a program or programs that, when executed, implement embodiments of the present invention. Accordingly, embodiments provide a program comprising code for implementing a system or method as claimed in any preceding claim and a machine readable storage storing such a program. Still further, embodiments of the present invention may be conveyed electronically via any medium such as a communication signal carried over a wired or wireless connection and embodiments suitably encompass the same.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The claims should not be construed to cover merely the foregoing embodiments, but also any embodiments which fall within the scope of the claims.

1. A method for generating a medical trial conclusion with respect to at least some participants in a medical trial, where medical trial data of the trial participants has been collected and the medical trial data collected comprises a data set pertaining to a first type of participant medical impact, the method comprising:
computer implemented analysis of the collected medical trial data of the medical trial participants to identify a first participant medical impact group by performing statistical clustering analysis for the medical trial participants in terms of participant medical impact as determined in accordance with the data set pertaining to the first type of participant medical impact, the first participant medical impact group corresponding to a statistical cluster so determined; and
generating a medical trial conclusion comprising an indication of the first participant medical impact group.

2. A method according to claim 1 where the medical trial data collected comprises at least one data set each pertaining to a respective type of participant trait, and the method comprises:
analysing the collected medical trial data of the medical trial participants to identify one or more first participant traits represented within the first participant medical impact group, as determined in accordance with the at least one data set each pertaining to a respective type of participant trait; and
where one or more first participant traits are identified, generating the medical trial conclusion to comprise an indication of at least one of the first participant traits.

3. A method according to claim 2 where the medical trial conclusion is generated to comprise an indication that individuals having a trait or traits consistent with one or more of the first participant traits are more likely to experience medical impact associated with the first participant medical impact group.

4. A method according to claim 1 where the medical trial data collected comprises a data set pertaining to a second type of participant medical impact.

5. A method according to claim 4 comprising:
computer implemented analysis of the collected medical trial data of trial participants in the identified first participant medical impact group to identify a first participant medical impact sub-group within that first participant medical impact group, by performing statistical clustering analysis for the participants in the identified first participant medical impact group in terms of participant medical impact as determined in accordance with the data set pertaining to a second type of participant medical impact, the first participant medical impact sub-group corresponding to a statistical cluster so determined;
and where the first participant medical impact sub-group has been identified, the medical trial conclusion comprises an indication of the first participant medical impact sub-group.

6. A method according to claim 5 comprising:
analysing of the collected medical trial data of the medical trial participants to identify one or more second participant traits represented within the identified first participant medical impact sub-group, as determined in accordance with the at least one data set each pertaining to a respective type of participant trait; and
where one or more second participant traits are identified, generating the medical trial conclusion to comprise an indication of at least one of the second participant traits.

7. A method according to claim 6 where the medical trial conclusion is generated to comprise an indication that individuals having a trait or traits consistent with one or more of the second participant traits are more likely to experience medical impact associated with the first participant medical impact sub-group.

8. A method according to claim 1 where performing the statistical clustering analysis comprises calculating a distance metric between each of the participants in the medical trial and each of the other participants in the medical trial, each distance metric indicating a relative degree of similarity of the respective pair of medical trial participants in respect of the data set pertaining to the first type of participant medical impact.

9. A method according to claim 8 where calculating each of the distance metrics comprises calculating a correlation coefficient between the respective pair of participants in the medical trial.

10. A method according to claim 9 where calculating the distance metrics comprises:
generating a first eigenspectrum of a correlation matrix comprising the correlation coefficients;
generating a second eigenspectrum of a random matrix having the same dimensions as the correlation matrix;
removing eigenvalues of the second eigenspectrum from the first eigenspectrum;
generating a final matrix using the remaining eigenvalues and using these as the distance metrics.

11. A method according to claim 10 where determination of the statistical clusters comprises using an algorithm that, given the distance metrics, seeks to maximise a function which indicates the degree of assortative grouping, modelling a baseline clustering configuration as a starting point and making random sequential adjustments to the configuration such that in each case the clusters are redefined so that one participant at a time is no longer in one cluster and is instead in another, analysing the effect of each adjustment on the function and where the function is larger, retaining the relevant adjustment and where the function is smaller, reversing the relevant adjustment.

12. A method according to claim 10 where the computation of the statistical clusters comprises additionally using the algorithm that seeks to maximise the function to model a different baseline clustering configuration as a starting point and making random sequential adjustments to the configuration such that in each case the clusters are redefined so that one participant at a time is no longer in one cluster and is instead in another, analysing the effect of each adjustment on the function and where the function is larger, retaining the relevant adjustment and where the function is smaller, reversing the relevant adjustment.

13. A method according to claim 1 where the medical trial data corresponds to only partial completion of the medical trial in that the medical trial participants constitute existing medical trial participants which are only a proportion of medical trial participants ultimately intended to form part of the medical trial, the medical trial data for the full medical trial ultimately being intended to additionally comprise medical trial data of further medical trial participants.

14. A method according to claim 13 comprising a computer implemented projection to extrapolate participant medical impact data known for the existing medical trial participants to account for at least one of the further medical trial participants and where the medical trial conclusion comprises a prediction dependent on the projection.

15. A method according to claim 14 where the extrapolation is performed in accordance with a continuous distribution of the corresponding participant medical impact among the existing medical trial participants.

16. A method according to claim 15 where the projection comprises performing a simulation instance realisation where an imaginary participant medical impact is generated for each of at least one of the further medical trial participants, the imaginary participant medical impacts being selected, in each case, at random and where the probability of any given imaginary participant medical impact arising in the random generation is consistent with the probability of the corresponding participant medical impact arising as determined by the prevalence or continuous distribution.

17. A method according to claim 16 where the projection comprises performing multiple instances of the simulation instance realisation and combining the imaginary participant medical impacts thereby generated for each simulation instance realisation with the participant medical impacts of the existing medical trial participants, wherein the mean of all such combinations is the prediction.

18. (canceled)

19. A method according to claim 1 comprising at least one of:
i) treating the medical trial participants in dependence on the medical trial conclusion;
ii) treating patients in dependence on the medical trial conclusion;
i) designing another medical trial in dependence on the medical trial conclusion;
ii) adapting the medical trial in dependence on the medical trial conclusion;
iii) halting the medical trial before its planned conclusion in dependence on the medical trial conclusion;
iv) making a determination on the success or failure of the medical trial in dependence on the medical trial conclusion; and
v) labelling, optionally automatically, a medication for treatment of one or more patients in dependence on the medical trial conclusion.

20-22. (canceled)

23. A medical trial conclusion generating system arranged to generate a medical trial conclusion with respect to at least some participants in a medical trial, where medical trial data of the medical trial participants has been collected and the medical trial data collected comprises a data set pertaining to a first type of participant medical impact, the system comprising:
an input arranged to receive the data set pertaining to the first type of participant medical impact;
a processor arranged to:
analyse the collected medical trial data of the trial participants to identify a first participant medical impact group by performing statistical clustering analysis for the participants in terms of participant medical impact as determined in accordance with the data set pertaining to the first type of participant medical impact, the first participant medical impact group corresponding to a statistical cluster so determined; and
an output arranged to output a medical trial conclusion comprising an indication of the first participant medical impact group.

24. (canceled)

25. A computer implemented method of projecting further data values for a partially complete data acquisition process pertaining to an entity or group of entities, the partially complete data acquisition process having acquired existing data values of a type and which are collated in an empirical distribution, the method comprising:
generating a continuous distribution by interpolating the empirical distribution; and
extrapolating in accordance with the continuous distribution to generate a simulation instance realisation comprising imaginary data values by:
generating random further data values of the type, where the probability of any given value of the further data arising in the random generation is consistent with the probability of the corresponding data value arising as determined by the continuous distribution,
where the imaginary data values are the projected further data values or the projected further data values are determined in dependence on the imaginary data values.
